(19) United States
(12) Patent Application Publication
(10) Pub. No.: US 2022/0015862 A1
(43) Pub. Date: Jan. 20, 2022

(54) DISPOSABLE CLEANING SYSTEM & METHOD FOR REUSABLE ENDOSCOPIC SYSTEMS

(71) Applicant: Covidien LP, Mansfield, MA (US)

(72) Inventors: Matthew A. Rootes, Crystal, MN (US); Nathan J. Knutson, Long Lake, MN (US)

(73) Assignee: Covidien LP, Mansfield, MA (US)

(21) Appl. No.: 17/347,319

(22) Filed: Jun. 14, 2021

Related U.S. Application Data
(60) Provisional application No. 63/053,436, filed on Jul. 17, 2020.

(51) Int. Cl. A61B 90/70; A61L 2/26; A61L 2/18; A61L 2/10; B08B 3/04; B08B 3/08; B08B 7/00

(52) U.S. Cl. CPC A61B 90/70; A61L 2/26; A61L 2/18; A61L 2/10; B08B 3/044; B08B 3/08; A61L 2202/11; A61B 2090/701; A61L 2202/24; A61L 2202/17; A61L 2202/122; A61L 2202/121; B08B 7/0057

(57) ABSTRACT

Systems and methods for cleaning a medical device include a disposable pouch having an interior surface defining a cavity and an agitator disposed within the cavity of the pouch.

### CROSS-REFERENCE TO RELATED APPLICATION

The present application claims the benefit of and priority to U.S. Provisional Application Ser. No. 63/053,436, filed on Jul. 17, 2020, the entire contents of which are hereby incorporated by reference herein.

### INTRODUCTION

This disclosure relates to surgical systems, and more particularly, to disposable cleaning systems and methods for reusable endoscopic systems.

### BACKGROUND

Advancements in endoscopic surgical procedures have given rise to the use of more complex and costly equipment, such as endobronchial ultra-sound catheters, endoscopes utilizing scanning lasers, multiple cameras, etc. With this increase in complexity and cost, it can be appreciated that medical facilities would prefer to re-use such equipment to minimize the capital investment associated with acquiring these surgical devices. Accordingly, the prevalence of single use surgical devices has decreased with the increased cost of such surgical devices, necessitating specialized disinfection, sterilization, and cleaning equipment to maintain the multi-use surgical devices utilized during endoscopic surgical procedures.

As can be appreciated, the cost to maintain, staff, and run the disinfection, sterilization, and cleaning equipment can be substantial, especially in facilities that perform numerous endoscopic surgical procedures per day. Further, such equipment often requires lengthy disinfection, sterilization, and/or cleaning procedures rending the surgical device unavailable for some time, thereby requiring medical facilities to either reduce the number of procedures performed per day, or acquire a significant number of identical surgical devices in order to perform the desired number of procedures.

The capital investment required to utilize the complex endoscopic surgical devices comes in two forms; the devices themselves and the corresponding equipment to disinfect, sterilize, clean, and maintain them. As the benefits of utilizing such complex medical devices during endoscopic procedures often outweigh the cost associated with the devices themselves, disposable cleaning systems may significantly reduce the cost associated with the reusable endoscopic surgical devices and may decrease the time required to disinfect, sterilize, and/or clean such equipment before being safely available to be used in another surgical procedure.

### SUMMARY

This disclosure is directed to a disposable cleaning system and methods of cleaning surgical instruments using a disposable cleaning system. The disposable cleaning system including a pouch having an interior surface defining a cavity and an agitator disposed within the cavity of the pouch. The agitator has an interior surface defining a channel along a length thereof. An exterior surface and the interior surface of the agitator defines an aperture in open communication with the channel and the aperture is configured to slidably receive a portion of a medical device therethrough and permit the medical device to be advanced within the channel to disinfect the portion of the medical device disposed within the channel.

In aspects, the agitator may include a spiral configuration terminating at a closed distal end portion.

In other aspects, the agitator may be formed from a resilient material such that as a medical device is advanced within the channel, the agitator transitions from the spiral configuration to a configuration conforming to a profile of the medical device.

In certain aspects, the channel of the agitator may include a chemical disinfectant disposed therein.

In other aspects, the aperture may include a penetrable seal configured to be penetrated by a portion of a medical device.

In certain aspects, the aperture may include a self-closing gland configured to be penetrated by a portion of a medical device.

In aspects, the disposable cleaning system may include a fluid reservoir disposed on the exterior portion of the agitator, the fluid reservoir having an inner surface defining a cavity having a chemical disinfectant disposed therein, wherein the cavity of the fluid reservoir is in fluid communication with the channel of the agitator.

In other aspects, the cavity of the fluid reservoir may include a burst seal disposed therein configured to selectively permit the flow of the chemical disinfectant from the cavity of the fluid reservoir into the channel of the agitator.

In aspects, the interior surface of the channel may include a plurality of protuberances disposed thereon configured to contact an exterior surface of the medical device as the medical device is advanced within the channel.

In certain aspects, the aperture of the agitator may include a coupling configured to selectively retain a portion of the medical device and inhibit removal of the medical device from the agitator.

In accordance with another aspect of this disclosure, a disposable cleaning system includes a pouch having an interior surface defining a cavity, an agitator disposed within the cavity of the pouch, and a controller operably coupled to a portion of the agitator. The agitator has an interior surface defining a channel along a length thereof. An exterior surface and the interior surface of the agitator defines an aperture in open communication with the channel and the aperture is configured to slidably receive a portion of a medical device therethrough and permit the medical device to be advanced within the channel to disinfect the portion of the medical device disposed within the channel. The controller includes a drive circuit, an electrical switch in electrical communication with the drive circuit, and a status indicator in electrical communication with the drive circuit.

In aspects, the disposable cleaning system may include a fluid reservoir disposed on the exterior portion of the agitator, the fluid reservoir having an inner surface defining a cavity having a chemical disinfectant disposed therein, wherein the cavity of the fluid reservoir is in fluid communication with the channel of the agitator.

In certain aspects, the cavity of the fluid reservoir may include a burst seal disposed therein configured to selectively permit the flow of the chemical disinfectant from the cavity of the fluid reservoir into the channel of the agitator.

In other aspects, the controller may include an ultrasonic transducer electrically coupled to the drive circuit, the ultrasonic transducer operably coupled to the channel of the agitator such that ultrasonic energy is permitted to propagate through the channel.

In certain aspects, the controller may include an ultra-violet (UV) light source electrically coupled to the drive circuit, the UV light source operably coupled to the channel of the agitator such that UV light is permitted to propagate through the channel.

In aspects, the inner surface of the agitator may include a reflective coating disposed thereon.

In accordance with another aspect of this disclosure, a method for cleaning a medical device includes breaking a seal of a pouch having an inner surface defining a cavity, advancing a distal end portion of a medical device through an aperture defined in an agitator, the agitator disposed within the cavity of the pouch, further advancing the medical device through the aperture and into a channel defined by an inner surface of the agitator, agitating the medical device within the channel to clean the medical device and retracting the medical device from the channel and aperture of the agitator.

In aspects, the method may include deforming a fluid reservoir having a chemical disinfectant disposed therein to cause the chemical disinfectant to transfer from the fluid reservoir and into the channel of the reservoir to clean the medical device.

In certain aspects, advancing the medical device within the channel of the agitator may cause the agitator to transition from a spiral configuration to a configuration conforming to a profile of the medical device.

In other aspects, agitating the medical device within the channel causes an exterior surface of the medical device to abut a plurality of protuberances disposed on the interior surface of the channel to clean the medical device.

### BRIEF DESCRIPTION OF THE DRAWINGS

Various aspects and features of the disclosure are described hereinbelow with references to the drawings, wherein:

FIG. 1 is a perspective view of a disposable cleaning system provided in accordance with the disclosure capable of being used with reusable endoscopic systems;

FIG. 2 is an interior view of the disposable cleaning system of FIG. 1;

FIG. 3 is an elevation view of an agitator of the disposable cleaning system of FIG. 1;

FIG. 4 is an elevation view of the agitator of FIG. 3 illustrating a portion of an endoscopic surgical device advanced therein;

FIG. 5 is an elevation, cross-sectional view of the agitator of FIG. 3 illustrating a portion of an endoscopic surgical device advanced therein;

FIG. 5A is an enlarged view of the area of detail indicated in FIG. 5;

FIG. 6 is a plan view of a coupling of the agitator of FIG. 3;

FIG. 6A is a cross-sectional view of the coupling of FIG. 6 taken along section line 6A-6A of FIG. 6;

FIG. 7 is an elevation, cross-sectional view of a fluid reservoir of the disposable cleaning system of FIG. 1;

FIG. 8 is a perspective view of a controller of the disposable cleaning system of FIG. 1;

FIG. 9 is a schematic view of the controller of FIG. 8;

FIG. 10 is a schematic view of the controller of FIG. 8 illustrating an ultrasonic transducer coupled thereto;

FIG. 11 is a schematic view of the controller of FIG. 8 illustrating an ultraviolet light (UV) emitted coupled thereto;

FIG. 12 is a schematic view of the controller of FIG. 8 illustrating a motorized conveyor system coupled thereto;

FIG. 13 is a schematic view of the controller of FIG. 8 illustrating a motorized inner tube coupled thereto;

FIG. 14 is a schematic view of the controller of FIG. 8 illustrating a motorized pump coupled thereto;

FIG. 15 is a flow chart illustrating a method of cleaning a medical device using the disposable cleaning system of FIG. 1;

FIG. 16 is a flow chart illustrating another method of cleaning a medical device using the disposable cleaning system of FIG. 1; and

FIG. 17 is a flow chart illustrating yet another method of cleaning a medical device using the disposable cleaning system of FIG. 1.

### DETAILED DESCRIPTION

This disclosure is directed to systems and methods for cleaning a medical device including a disposable cleaning system having a pouch with an agitator disposed within an interior portion thereof. The agitator includes a channel formed therein configured to slidably receive a portion of a used medical device therein to clean or otherwise disinfect the used medical device for use in a subsequent surgical procedure. The agitator includes an aperture sealed by a penetrable seal such that the channel of the agitator is maintained in a sterile state until the distal portion of the medical device penetrates the penetrable seal and enters the channel. The agitator is disposed in a coiled or otherwise spiral like configuration and is formed from a resilient material such that as the medical device is advanced within the channel of the agitator, the agitator is uncoiled or otherwise caused to conform to the configuration of the medical device (e.g., linear, curvate, etc.) The spiral like configuration causes the medical device to abut or otherwise drag against an interior surface of the channel to enhance the cleaning ability of the agitator.

A chemical disinfectant may be disposed within the channel of the agitator to clean or otherwise disinfect the medical device 200 as it is advanced within the channel. In embodiments, the chemical disinfectant may be disposed in a fluid reservoir located outside of the channel and in fluid communication therewith. In this manner, the fluid reservoir may be compressible or deformable such that the fluid disposed within the fluid reservoir may be expelled therefrom and fill the channel of the agitator. In embodiments, the fluid reservoir includes a burst seal to inhibit the chemical disinfectant stored therein from entering the channel of the agitator until use. In this manner, as the fluid reservoir is compressed the pressure within the fluid reservoir increases until the burst seal ruptures, permitting the chemical disinfectant to flow into the channel of the agitator.

The disposable cleaning system may include a controller having a printed circuit board (PCB) with a plurality of electrical components disposed thereon, such as one or more drive circuits, an energy storage device, a light emitting diode (LED), and a button or switch. The drive circuits include a processor and a memory storing instructions, which when executed by the processor cause the processor to perform one or more functions, such as initiating a timing program. The timing program measures the amount of time elapsed from the time at which the button was toggled until a predetermined amount of time has elapsed, at which point the processor causes the LED to turn off or change colors to indicate that the cleaning process has been completed. In embodiments, the controller may include an ultrasonic transducer, an ultraviolet (UV) light emitter, a motorized conveyor belt system, a rotating interior tube, or a pump that can be actuated by toggling the button to execute one or more programs to cause the ultrasonic transducer, the UV light emitter, the motorized conveyor belt system, the rotating interior tube, or pump to agitate or otherwise clean and disinfect the medical device that has been advanced within the channel of the agitator.

In use, after a surgical procedure has been performed, the pouch is opened to expose the agitator. A distal portion of the used medical device is advanced within the aperture of the agitator and penetrates the penetrable seal to enter the channel of the agitator. The medical device is further advanced within the channel of the agitator until the medical device is fully advanced therein. At this point, the medical device is retracted from the channel of the agitator, and thereafter, from the aperture to disinfect or otherwise clean the medical device. The aperture may include a coupling to selectively couple the medical device to the agitator an inhibit removal of the medical device without further manipulation of the medical device. In an instance where the disposable cleaning system includes a fluid reservoir, the fluid reservoir is compressed to cause the burst seal to rupture and permit the chemical disinfectant to flow within the channel of the agitator to aid in disinfecting and/or cleaning the medical device.

In a case where the disposable cleaning system includes a controller, the button is depressed to cause the processor to initiate the timing program and cause the LED to illuminate to indicate that the cleaning process has begun. After the predetermined amount of time has elapsed, the timing program causes the LED to shut off to indicate that the cleaning process is completed and thereafter, the medical device is removed from the agitator and the pouch and agitator are discarded. The use of the disposable cleaning system having the ultrasonic transducer, the UV light emitter, the motorized belt system, the rotating inner tube, and/or the pump is similar in that the button is depressed to cause the ultrasonic transducer, the UV light emitter, the motorized belt system, rotating inner tube, and/or pump to activate and agitate the medical device advanced within the channel of the agitator. In embodiments, the button may be utilized to increase the amount of time the program runs, increase or decrease the amount of energy generated by the ultrasonic transducer, the UV light emitter, the motorized belt system, rotating inner tube, and/or pump, and/or combinations thereof.

Embodiments of the disclosed disposable cleaning systems are described in detail with reference to the drawings, in which like reference numerals designate identical or corresponding elements in each of the several views.

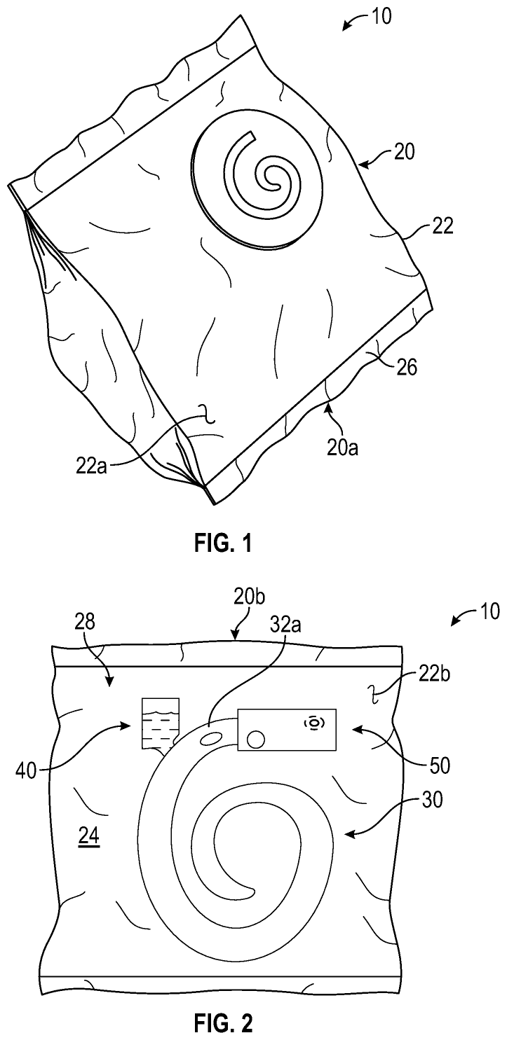

With reference to FIGS. 1 and 2, a disposable cleaning system for use with endoscopic system and generally identified by reference numeral 10. The disposable cleaning system 10 includes a pouch or bag 20, an agitator 30, a fluid reservoir 40, and a controller 50.

The pouch 20 is formed from a resilient material and in embodiments, may be formed from oriented polypropylene film (OPP), polyethylene terephthalate (PET), flashspun high-density polyethylene fibers (e.g., Tyvek®), high-density polyethylene (HDPE), metallised films (e.g., metallised PET, metallised OPP, metallised polypropylene (PP), metallised nylon, metallised polyethelyne, etc.), a foil, etc. In one non-limiting embodiment, the pouch 20 is formed from metallised PET (MET-PET).

The pouch 20 includes a sheet 22 having an exterior surface 22a and an opposite, interior surface 22b. The interior surface 22b of the sheet 22 defines a cavity 24 therein that is configured to receive various components of the disposable cleaning system 10 as will be described in further detail hereinbelow. Although generally illustrated as having a single sheet 22 forming a cylindrical profile (e.g., contiguous about its circumference), it is contemplated that the pouch 20 may be formed from two or more sheets bonded to one another adjacent an exterior perimeter of each (e.g., four seals are formed to seal the pouch 20). In embodiments, the single sheet 22 may be extruded as a single cylindrical component (e.g., having no longitudinal seals) or may be a single sheet folded over and sealed or otherwise joined along a longitudinal axis thereof (e.g., three total seals to form the pouch 20).

The interior surface 22b of the sheet 22 of the pouch 20 is bonded or otherwise sealed at a first seal location 26 adjacent a distal end portion 20a of the pouch 20 using any suitable means, such as heat sealing, adhesives, a lap seal, a fin seal, etc. In one non-limiting embodiment, the first seal location 26 is formed using a procedure capable of forming a hermetic seal. As can be appreciated, by sealing the pouch 20 only at the first seal location 26, the cavity 24 defines a mouth or opening 28 at a proximal end portion 20b (e.g., an end of the pouch 20 that is opposite to the distal end 20a thereof). As can be appreciated, once all of the components described herein have been disposed within the cavity 24 of the pouch 20, the pouch 20 is sealed at a second seal location 26a adjacent the opening 28 of the cavity 24 to seal or otherwise hermetically seal the pouch 20 and inhibit the ingress of contaminants into the cavity 24.

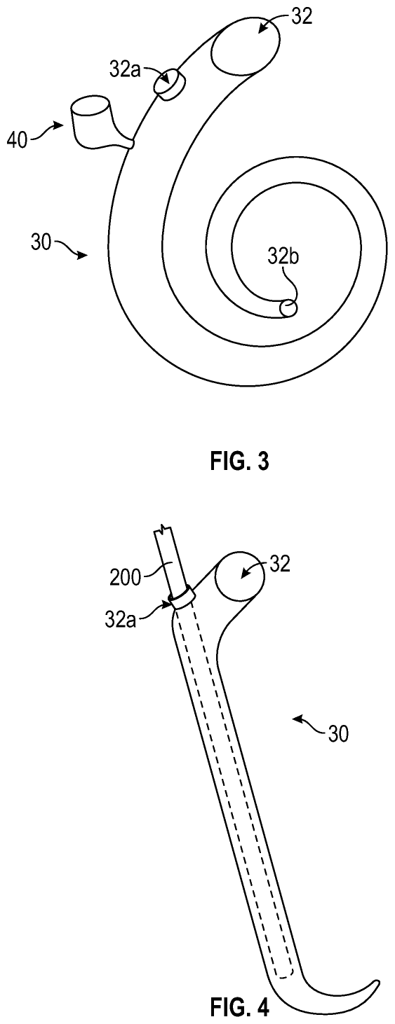

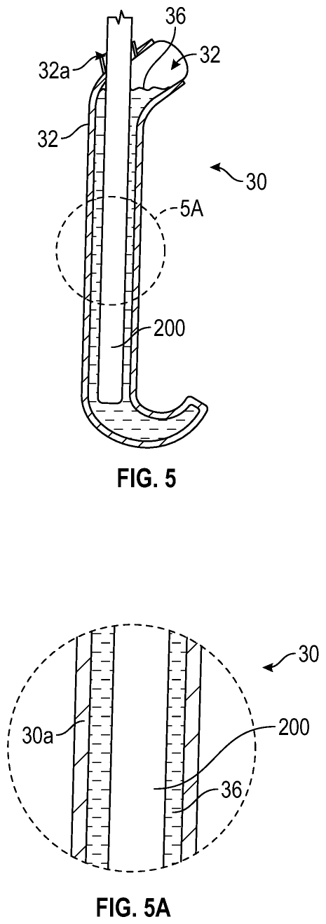

Turning now to FIGS. 3-6A and with continued reference to FIG. 2, the agitator 30 is disposed within the cavity 24 of the pouch 20 and includes a generally cylindrical profile having a circular cross-section, although it is contemplated that the agitator 30 may include any suitable cross-section, such as square, oval, hexagonal, octagonal, triangular, etc. In embodiments, the cross-sectional dimensions of the agitator decrease in size such as a funnel or conical profile is formed. The agitator 30 includes an inner surface 30a (FIG. 5) defining a channel 32 therein having an open proximal end portion or aperture 32a and an opposite, closed distal end portion or distal end wall 32b. In this manner, the aperture 32a is configured to selectively receive a portion of a medical device 200 therein and the distal end wall 32b is configured to inhibit the medical device 200 from exiting other otherwise passing entirely through the channel 32 of the agitator 30, as will be described in further detail hereinbelow. Although generally described as terminating at an end wall 32b, it is contemplated that the distal portion of the agitator may taper in a longitudinal direction until the channel 32 of the agitator 30 is closed.

It is contemplated that the channel 32 of the agitator 30 may include an inner diameter configured to receive a variety of medical devices, such as an endoscope, a bronchoscope, an endoscopic ultrasound (EUS) device, an endobronchial ultrasound (EBUS) device, an endoscopic vessel sealer, etc. In embodiments, it is contemplated that the channel 32 of the agitator 30 may include an inner diameter suitable for specific medical devices, and in embodiments, the inner diameter of the channel 32 of the agitator 30 is approximately 1.9 mm to 2.0 mm.

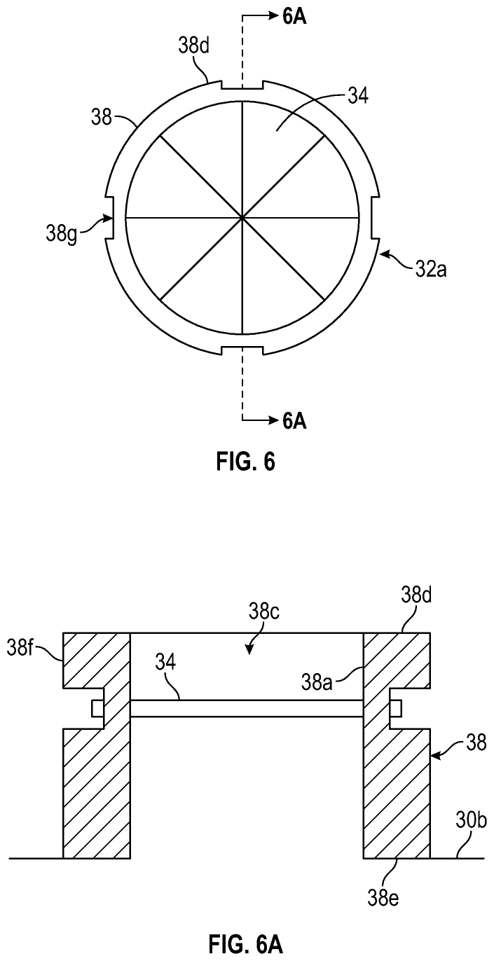

The agitator 30 may be disposed in a coiled or otherwise spiral like configuration and is formed from a resilient material capable of permitting the agitator 30 to uncoil or otherwise be caused to conform to a linear or straight configuration as the medical device 200 is advanced therein. Alternatively, the agitator 30 remain coiled and the medical device, being flexible, passes through the coiled configuration. Still further, the agitator 30 may be formed as a long straight catheter configured to receive the medical device. As will be appreciated other shapes may also be employed without departing from the scope of the disclosure. In embodiments, the agitator 30 is formed from a biologically inert material having resilient characteristics, such as a polymer, a composite, etc. In one non-limiting embodiment, the agitator is formed from a silicone using an injection molding process. The agitator 30 includes a septum or other suitable selectively or permanently penetrable seal 36 (FIGS. 6 and 6A) over or adjacent the aperture 32a such that the channel 32 of the agitator 30 is hermetically sealed. In embodiments, the septum 34 may be formed from a foil, polymer, etc. capable of being penetrated by the medical device 200, as will be described in further detail hereinbelow.

With reference to FIGS. 6 and 6A, a coupling 38 for selectively retaining a portion of the medical device 200 is disposed on an exterior surface 30b of the agitator 30 and includes a generally cylindrical profile, although any suitable profile is contemplated. The coupling includes an inner surface 38a defining a channel 38c through opposed upper and lower surfaces 38d and 38e, respectively. The channel 38c of the coupling 38 is on the disposed on the exterior surface 30b of the agitator 30 such that the channel 38c is in open communication with the aperture 32a. In this manner, a portion of the medical device 200 is permitted to be advanced within the channel 38c of the coupling 38 and thereafter, be advanced within the aperture 32a, as will be described in further detail hereinbelow.

An outer surface 38f of the coupling 38 defines a lock 38g or other suitable device capable of selectively securing a portion of the medical device 200 thereto to selectively inhibit removal of the medical device 200 from the coupling 38 during a cleaning or disinfecting process, as will be described in further detail hereinbelow. In embodiments, the coupling 38 may include a tab (not shown) or other suitable device capable of emitting an audible click or other suitable indicator to a user to indicate that the medical device 200 has been fully secured to the coupling 38.

Turning to FIG. 5, the inner surface 30a of the agitator 30 is coated with a chemical disinfectant 36 or other suitable compound, fluid, or gel capable of disinfecting contaminated medical devices, such as glutaraldehyde solutions, hydrogen peroxide solutions, ortho-phthalaldehyde (OPA) solutions, peracetic acid-hydrogen peroxide solutions, sodium hypochlorite solutions, etc. In this manner, after the inner surface 30a of the agitator 30 is coated with the chemical disinfectant 36, the aperture 32a is sealed using the septum 34 to ensure the channel 32 is sterile or otherwise disinfected. In embodiments, the inner surface 30a may be smooth, may include a plurality of protuberances, fins, bristles, crenellations, etc., or may include a flocking or other unidirectional or multidirectional component capable of contacting or otherwise scrubbing the medical device 200 as the medical device 200 is advanced within the channel 32.

In embodiments, the chemical disinfectant 36 is disposed within the fluid reservoir 40 disposed on the exterior surface 30b of the agitator 30. It is contemplated that the fluid reservoir 40 may be affixed to agitator 30 using any suitable means, such as ultrasonic welding, adhesives, heat sealing, etc. In embodiments, the fluid reservoir 30 may be integrally formed with the agitator 30 and portions of the fluid reservoir 40 may be sealed in a manner similar to that of the pouch 20 (e.g., a perimeter of the fluid reservoir 40 may be sealed), described in detail hereinabove.

The fluid reservoir 40 is a pouch or other suitable vessel having an interior cavity 42 capable of storing or otherwise receiving a fluid therein. In one non-limiting embodiment, the fluid reservoir 40 is a pouch formed from a resilient material capable of being deformed or otherwise collapsed to cause the chemical disinfectant 36 stored therein to be expelled from the interior cavity 42. In this manner, a portion of the interior cavity 42 defines an aperture 46 in fluid communication therewith through which the chemical disinfectant 36 stored within the interior cavity 42 may be expelled. To enable the chemical disinfectant 36 stored within the interior cavity 42 of the pouch 40 to be transferred to the channel 32 of the agitator 30, a bore 30c is defined through the interior and exterior surfaces 30a, 30b of the agitator 30 that is in fluid communication with the channel 32 of the agitator and the interior cavity 42 of the fluid reservoir 40.

In embodiments, the fluid reservoir 40 may include a frangible or burst seal 48 disposed within the aperture 46 to inhibit the chemical disinfectant 36 stored within the interior cavity 42 from being expelled therefrom unless the burst seal 48 has been broken or otherwise breached. The burst seal 48 is configured to burst or otherwise be breached due to an increase in pressure within the interior cavity 42 of the fluid reservoir 40 caused by deformation (e.g., decrease in volume) of the fluid reservoir. In this manner, a user may apply pressure or otherwise deform the fluid reservoir 40 to cause an increase in pressure within the interior cavity 42 of the fluid reservoir and cause the burst seal 48 to burst and permit the chemical disinfectant 36 to flow from the interior cavity 42 and into the channel 32 of the agitator 30. It is contemplated that the burst seal 48 may be formed from a metallic material (e.g., foil, etc.), a nonmetallic material (e.g., a polymer, a composite, etc.), an ultrasonic welded seal, a heat seal, an adhesive seal, or any other suitable material capable of rupturing or otherwise fracturing due to a pressure being applied thereto. As can be appreciated, the burst pressure (e.g., the pressure required to cause the burst seal 48 to rupture) of the burst seal 48 must be lower than that of the material from which the fluid reservoir 40 is formed to ensure that the burst seal 48 ruptures before a failure of the fluid reservoir 40.

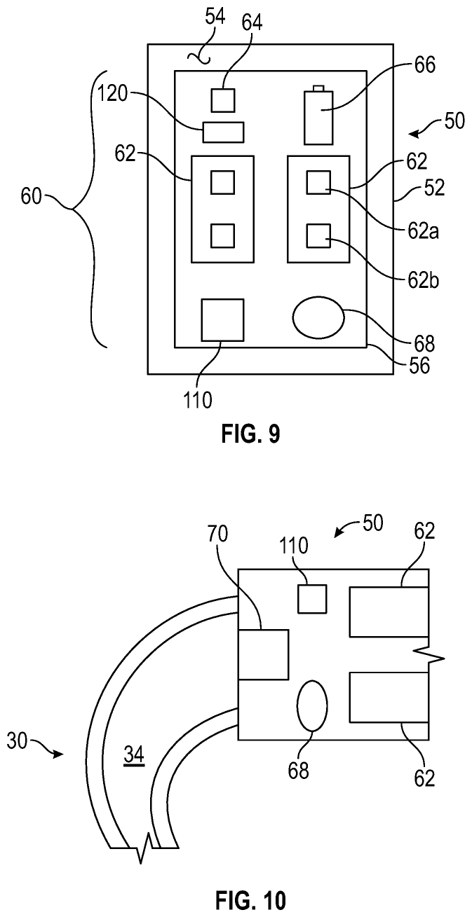

Returning to FIG. 2 and with additional reference to FIGS. 8 and 9, the controller 50 includes a housing 52 having an interior surface defining a chamber 54 therein. A printed circuit board (PCB) 56 is disposed within the chamber 54 of the housing 52 and includes one or more electrical components 60 disposed thereon. The electrical components 60 are disposed on the PCB 56 and include one or more drive circuits 62, a light emitting diode (LED) 64, an energy storage device 66, and a button or button 68.

The one or more drive circuits 62 are electrically coupled to the PCB 56 and may include a processor 62a and a memory 62b storing instructions, which when executed by the processor 62, cause the processor 62 to perform one or more functions, as will be described in further detail hereinbelow. The memory 62b may include any non-transitory computer-readable storage media for storing data and/or software that is executable by the processor 62, e.g., solid-state, volatile, non-volatile, removable, and non-removable. The LED 64 may be any suitable LED capable of emitting light, and in embodiments, may be an Organic LED, a single-color LED, a multi-color LED (e.g., bi-color, RGB Tri-color, etc.), a surface mount LED, through-hole LED, etc. The LED 64 is in electrical communication with one or more of the one or more drive circuits 62 such that the one or more drive circuits 62 cause the LED 64 to emit light to provide an indication of certain conditions, as will be described in further detail hereinbelow. Although generally described as being a single LED, it is contemplated that the electrical components may include a plurality of LEDs depending upon the design requirements of the disposable cleaning system 10.

The energy storage device 66 is electrically coupled to the PCB 56 and may be any energy storage device 66 capable of providing energy to the one or more drive circuits 62 and the LED 64, such as a battery, etc. In embodiments, the controller 50 may include a removable tab or film (not shown) interposed between the energy storage device 66 and the PCB 56 to inhibit electrical continuity between the energy storage device 66 and PCB 56 and inhibit draining of the energy storage device 66 during storage or while the disposable cleaning system 10 is not in use. In this manner, the tab can be removed to cause the energy storage device 66 and the PCB 56 to be electrically coupled to one another at a desired time (e.g., right before use of the disposable cleaning system 10) to ensure that the energy storage device 66 includes the required energy for the duration of use of the disposable cleaning system 10.

The button 68 is electrically coupled to the PCB 56 and is used to selectively inhibit the transmission of energy from the energy storage device 66 to the one or more drive circuits 62 (e.g., toggle the controller 50 to an on or off condition). In embodiments, the button 68 may be utilized solely to toggle the controller to an on condition. The button 68 may be a mechanical switch (e.g., contact switch), a capacitive switch, etc., although it is contemplated that the button 68 may be any suitable button capable of being actuated to alter a state or condition of the controller 50. In one non-limiting embodiment, the button 68 is configured to toggle the controller from an “off” position to an “on” position.

In embodiments, the memory 62b of the one or more drive circuits 62 stores instructions, which when executed by the processor 62a, cause the processor to initiate a timing program. The timing program is configured to measure an elapsed time from the depression of the button 68 and illuminate the LED 64 when a predetermined amount of time has elapsed. The predetermined amount of time may be an amount of time determined to effectively disinfect or otherwise clean the medical device 200 advanced within the channel 32 of the agitator 30. In this manner, after the medical device 200 has been fully advanced within the channel 32 of the agitator 30, the button 68 may be depressed to initiate a counter. Once the predetermined amount of time has elapsed, the processor causes the LED 64 to illuminate to indicate that the medical device 200 has been properly disinfected and may be removed from the channel 32 of the agitator 30. It is contemplated that the LED 64 may transition from an inactive state (e.g., not illuminated) to an active state (e.g., illuminated), however, it is envisioned that the LED 64 may transition from a first color (e.g., red) once the timing program is initiated to a second color (e.g., green) when the predetermined amount of time has elapsed. In one non-limiting embodiment, the electrical components 60 may include a speaker, buzzer, or transducer (not shown) to emit an audible alarm when the predetermined amount of time has elapsed and/or when the button 68 is actuated. In embodiments, the button 68 is configured to control or otherwise alter the performance of the disposable cleaning system 10. In this manner, the button 68 causes the processor 62 to increase or decrease the predetermined amount of time before the LED 64 is caused to be illuminated. It is envisioned that the processor 62a may cause the LED 64 to blink or otherwise illuminate in a predetermined pattern to indicate various selections, such as an increase or decrease in time.

With reference to FIG. 9, it is contemplated that the controller 50 may include a wireless communication module 110 operably coupled to at least one of the one or more drive circuits 62, and in embodiments, may be operably coupled to a stand alone drive circuit (not shown). The wireless communication module 110 wirelessly transmits data using any suitable wireless protocol capable of wirelessly transmitting data either in real time or on demand, such as those conforming to IEEE 802, Zigbee, Bluetooth, or the like. In one non-limiting embodiment, the wireless communication module 110 may include a network connection interface, such as a wireless transceiver, etc.

The wireless communication module 110 is configured to wirelessly communicate with a corresponding wireless communication module or network interface operably coupled to the medical device 200 advanced within the channel 32 of the agitator 30. In this manner, the wireless communication module 110 can cause a use counter (e.g., a counter configured to count the number of times a device has been utilized and/or cleaned) disposed within the medical device 200 to increment by one (e.g., from “0” to “1,” from “1” to “2,” etc.) to indicate that the device has been utilized during a surgical procedure and has been cleaned and/or sterilized.

It is envisioned that the controller 50 may include an RFID reader 120 operably coupled to at least one of the one or more drive circuits 62 that is configured to read a corresponding RFID chip disposed within the medical device 200 advanced within the channel 32 of the agitator 30. It is contemplated that the RFID reader 120 may read the RFID chip of the medical device 200 and compare the data stored on the RFID chip with a database stored on the memory 62b. In this manner, the controller may inhibit, or permit, operation of the disposable cleaning system 10 depending upon whether the data stored on the RFID chip correlates to data stored on the memory 62b.

With reference to FIG. 10, it is contemplated that the electrical components 60 may include an ultrasonic transducer 70 electrically coupled to the PCB board 56 and at least one of the one or more drive circuits 62. The ultrasonic transducer is electromagnetically coupled to the channel 32 of the agitator 30 such that ultrasonic energy emitted from the ultrasonic transducer 70 is propagated through the channel 32 and agitates an outer surface of the medical device 200 advanced within the channel 32. As can be appreciated, the ultrasonic energy emitted from the ultrasonic transducer 70 may also agitate the chemical disinfectant 36 disposed within the channel 32 of the agitator 30 to further clean and/or disinfect the medical device 200. Although generally described as being an ultrasonic transducer, it is contemplated that the ultrasonic transducer 70 may be any suitable device capable of emitting electromagnetic energy or wave propagation to agitate the medical device 200, chemical disinfectant, or combinations thereof. In embodiments, the electrical components may include a vibration motor (e.g., eccentric rotating mass vibration motor (ERM), a speaker, a micro-electromechanical system (MEMS), etc. to agitate the chemical disinfectant 36, the agitator 30, the medical device 200, or combinations thereof. As can be appreciated, one or more of an ultrasonic transducer 70, a vibration motor, a speaker, a MEMS, etc. or combinations thereof. In embodiments, the button 68 is configured to alter the intensity in which the ultrasonic transducer 70 operates, thereby altering the intensity at which the medical device 200 is cleaned. It is envisioned that the processor 62a may cause the LED 64 to blink or otherwise illuminate in a predetermined pattern to indicate various selections, such as an increase or decrease in intensity of the ultrasonic energy emitted by the ultrasonic transducer 70.

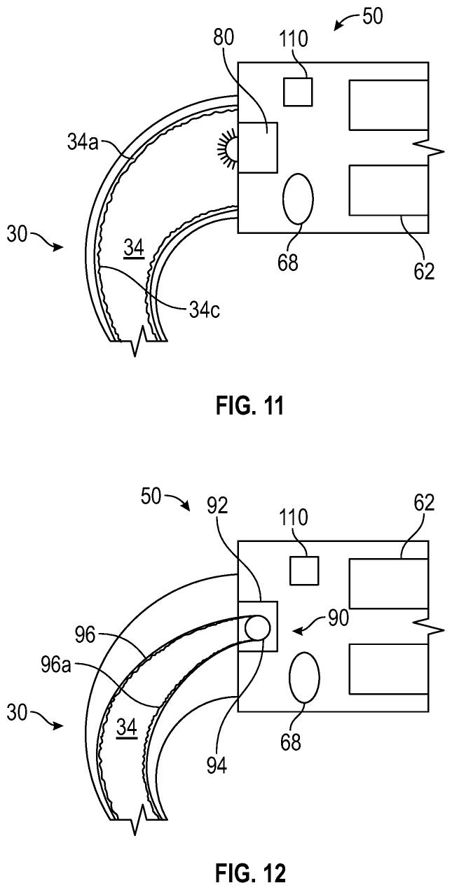

With reference to FIG. 11, it is contemplated that the electrical components 60 may include a Ultra Violet (UV) light emitter 80 electrically coupled to the PCB 56 and at least one of the one or more drive circuits 62. The UV light emitter 80 is in electromagnetic communication with the channel 32 of the agitator 30 such that UV light emitted from the UV light emitter 80 is propagated through the channel 32 to sterilize or otherwise disinfect the medical device 200 advanced therein. In embodiments, the inner surface 34a of the channel 32 may be coated with a reflective coating 34c capable of reflecting the UV light propagating within the channel 32 towards the medical device 200 and along the length of the channel 32. In embodiments, the button 68 is configured to alter the intensity of the UV light, a pattern in which the UV light is emitted, etc. thereby altering the intensity at which the medical device 200 is cleaned. It is envisioned that the processor 62a may cause the LED 64 to blink or otherwise illuminate in a predetermined pattern to indicate various selections, such as an increase or decrease intensity of the UV light emitted from the UV light emitter 80, various patterns of UV light emitted from the UV light emitter 80, etc.

Turning to FIG. 12, it is envisioned that the electrical components 60 may include a motorized conveyor belt system 90 having an electric motor 92, a pully 94, and a belt 96. The electric motor 92 is electrically coupled to the PCB 56 and at least one of the one or more drive circuits 62. The pulley 94 is operably coupled to an output shaft (not shown) of the electrical motor 92 such that rotation of the output shaft effectuates a corresponding rotation of the pulley 94. The belt 96 is a contiguous belt having a length corresponding to a length of the channel 32 of the agitator. In this manner, as the medical device 200 is advanced within the channel 32, the belt 96 encompasses the medical device 200 such that a plurality of protuberances 96a defined on an interior surface 96b of the belt 96 abut or bush against an exterior surface of the medical device 200 to mechanical scrub or otherwise clean the medical device 200. It is envisioned that the pully 94 may effectuate rotation of the belt 96 using any suitable means, such as friction, lugs, etc. Although generally described as being coupled to the pulley 94, it is contemplated that the belt 96 may be operably coupled to the output shaft of the electric motor 92 using any suitable means capable of transmitting the rotation of the output shaft of the electric motor 92 to the belt 96. In embodiments, the button 68 is configured to alter the intensity in which the electric motor 92 operates, thereby altering the intensity at which the medical device 200 is cleaned. It is envisioned that the processor 62a may cause the LED 64 to blink or otherwise illuminate in a predetermined pattern to indicate various selections, such as an increase or decrease in speed at which the electric motor 92 operates.

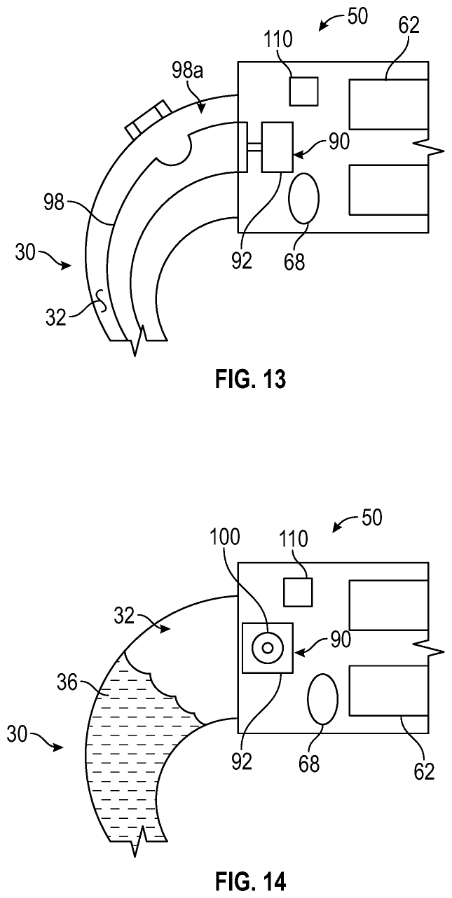

With reference to FIG. 13, it is contemplated that the belt 96 of the motorized conveyor belt system 90 may be replaced with an interior tube 98 formed from a resilient material and disposed within the channel 32 of the agitator 30. A proximal portion 98a of the interior tube 98 is operably coupled to the output shaft (not shown) of the electrical motor 92 such that rotation of the output shaft effectuates a corresponding rotation of the interior tube 98 within the channel 32 of the agitator 30. It is contemplated that an interior surface 98b of the interior tube 98 may include a plurality of protuberances (not shown) or other suitable components capable of abutting or otherwise brushing against the exterior surface of the medical device 200 as the interior tube 98 rotates thereabout. It is contemplated that the interior tube 98 may be operably coupled to the output shaft of the electric motor 92 using any suitable means, such as adhesives, mechanical fasteners, friction, couplings, etc.

With reference to FIG. 14, it is envisioned that the output shaft of the electric motor 92 may be operably coupled to an impeller or pump 100 in fluid communication with the chemical disinfectant 36 disposed within the channel 32 of the agitator 30. In this manner, actuation of the pump 100 effectuates flow of the chemical disinfectant 36 within the channel 32 to aid in cleaning or sterilizing of the medical device 200 advanced within the channel 32. As can be appreciated, the pump 100 may be any suitable pump capable of effectuating a flow of fluid. In one non-limiting embodiment, the pump 100 may be an air pump configured to circulate air within the channel 32 of the agitator 30. In embodiments, the button 68 is configured to alter the intensity in which the pump 100 operates, thereby altering the intensity at which the medical device 200 is cleaned. It is envisioned that the processor 62a may cause the LED 64 to blink or otherwise illuminate in a predetermined pattern to indicate various selections, such as an increase or decrease in speed at which the electric motor 92 operates.

Although generally described as being individual embodiments, it is contemplated that the controller 50 may include one or more of the ultrasonic transducer 70, the UV light emitter 80, the conveyor belt system 90, the rotating interior tube 98, the pump 100, or combinations thereof.

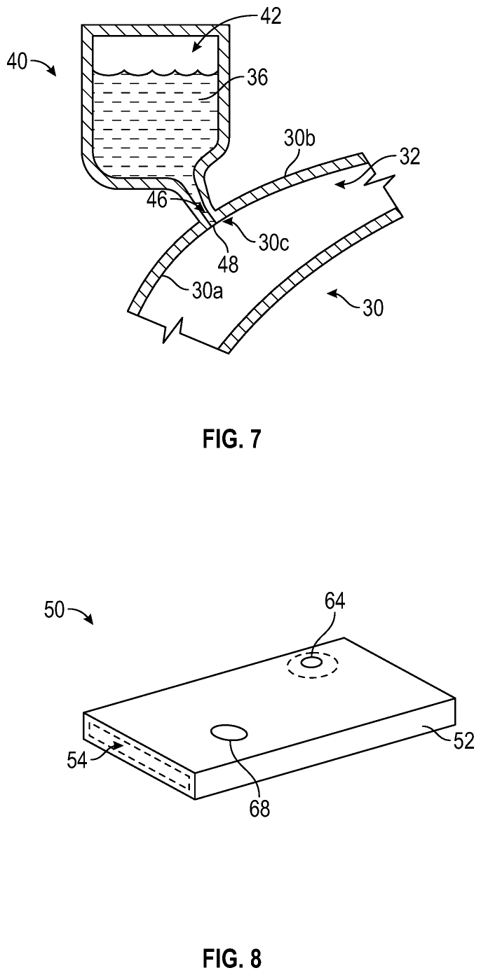

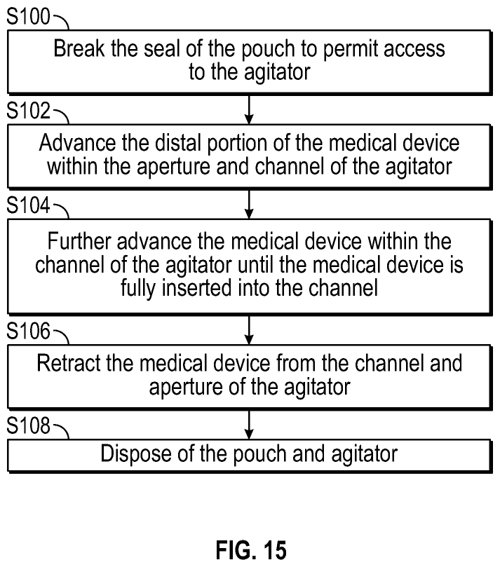

With reference to FIGS. 1-15, in operation, after a surgical procedure has been performed and there is no longer a need to utilize the medical device 200, in step S100, the seal at the first or second seal locations 26, 26a is broken to open the pouch 20 and permit access to the agitator 30. In step S102, the distal portion of the medical device 200 is advanced within the aperture 32a of the agitator 30, and thereafter, within the channel 32 of the agitator 30. In step S104, the medical device 200 is further advanced within the channel 32 of the agitator until the medical device 200 is fully inserted within the channel 32. At this point, in step 106, the medical device 200 may be retracted from the channel 32 of the agitator, and thereafter, from the aperture 32a, at which point the medical device 200 has been cleaned or otherwise disinfected and is once again ready to be utilized during a subsequent surgical procedure. In embodiments, an optional step S105 may be employed where the medical device 200 is manually agitated within the channel by partially and repeatedly retracting and/or rotating the medical device 200 within the channel 32 of the agitator 30 to clean the medical device 200. Manual agitation can be particularly efficiently achieved when the inner surface 30a of the channel 32 includes a plurality of protuberances, fins, bristles, crenellations, or other unidirectional or multidirectional components. These protuberances contact the surface of the medical device 200 as the medical device 200 is advanced within the channel 32 to remove contaminants and clean the surface of the medical device 200. After the cleaning process has been completed, in step S108, the pouch 20 and the agitator 30 are discarded.

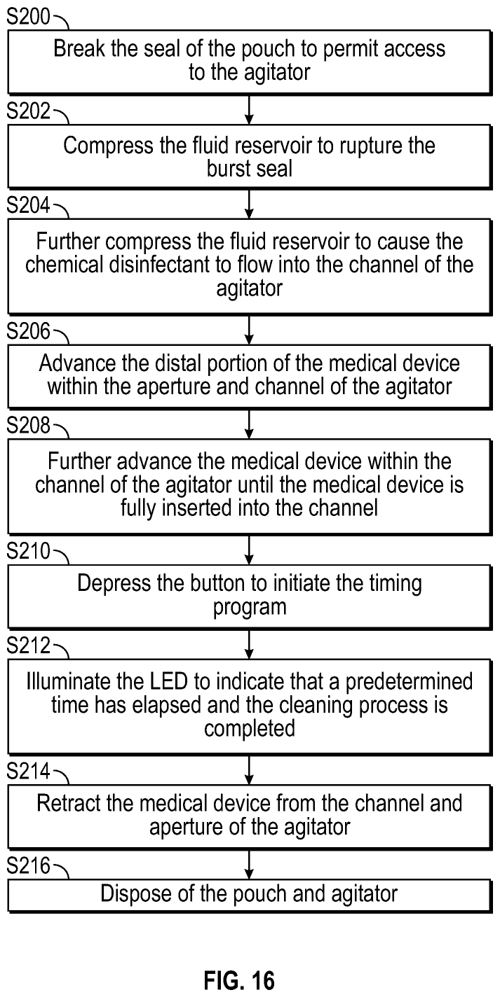

With reference to FIGS. 1-14 and 16, where the disposable includes a fluid reservoir 40 and a controller 50, in step S200, the seal at the first or second seal locations 26, 26a is broken to open the pouch 20 and permit access to the agitator 30, the fluid reservoir 40, and the controller 50. In step S202, the fluid reservoir 40 is compressed or otherwise deformed to cause a pressure within the interior cavity 42 of the fluid reservoir 40 to increase and cause the burst seal 48 to rupture. In step S204, the fluid reservoir is further compressed to cause the chemical disinfectant 36 stored within the interior cavity 42 of the fluid reservoir 40 to be expelled and fill the channel 32 of the agitator 30. At this point, in step S206, the distal portion of the medical device 200 is advanced within the aperture 32a of the agitator 30, and thereafter, within the channel 32 of the agitator 30. In step S208, the medical device 200 is further advanced within the channel 32 of the agitator until the medical device 200 is fully inserted within the channel 32. In embodiments where the agitator 30 includes a coupling 38, the medical device 200 is inserted within the channel 32 of the agitator 30 until the medical device 200 actuates a tab or other suitable device (not shown) capable of generating an audible click or the like to indicate that the medical device 200 has been fully inserted within the channel 32 of the agitator 30. At this point, the medical device 200 is coupled to the coupling 38 inhibiting removal therefrom without additional manipulation of the medical device 200.

In step S210, the button 68 is depressed to cause the processor 62a to initiate the timing program stored on the memory 62b of the one or more drive circuits 62 and cause the LED 64 to illuminate to indicate that the cleaning process has begun. In step S212, after a predetermined amount of time has elapsed, the timing program causes the LED 64 to shut off (e.g., stop illuminating light) or change color (e.g., from red to green, etc.). At this point, in step S214, the medical device 200 is rotated to disengage from the coupling 38, and thereafter, is removed from the channel 32 of the agitator 30, and thereafter, from the aperture 32a, at which point the medical device 200 has been cleaned or otherwise disinfected and is once again ready to be utilized during a subsequent surgical procedure. After the cleaning process has been completed, in step S216, the pouch 20 and the agitator 30 are discarded.

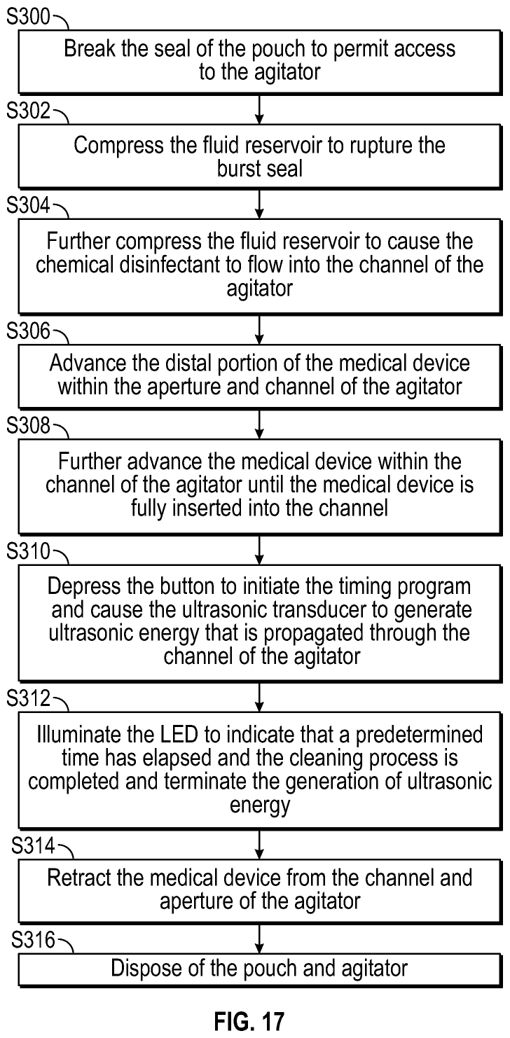

With reference to FIGS. 1-14 and 17, the operation of the disposable cleaning system 10 including an ultrasonic transducer 70, a UV light emitter 80, a motorized belt system 90, a rotating inner tube 98, or a pump 100 is described. The use of the disposable cleaning system 10 having each is substantially similar, and thus, only the use of the disposable cleaning system 10 having an ultrasonic transducer 70 will be described herein in the interest of brevity.

In step S300, the seal at the first or second seal locations 26, 26a is broken to open the pouch 20 and permit access to the agitator 30, the fluid reservoir 40, and the controller 50. In step S302, the fluid reservoir 40 is compressed or otherwise deformed to cause a pressure within the interior cavity 42 of the fluid reservoir 40 to increase and cause the burst seal 48 to rupture. In step S304, the fluid reservoir is further compressed to cause the chemical disinfectant 36 stored within the interior cavity 42 of the fluid reservoir 40 to be expelled and fill the channel 32 of the agitator 30. At this point, in step S306, the distal portion of the medical device 200 is advanced within the aperture 32a of the agitator 30, and thereafter, within the channel 32 of the agitator 30. In step S308, the medical device 200 is further advanced within the channel 32 of the agitator until the medical device 200 is fully inserted within the channel 32. In embodiments where the agitator 30 includes a coupling 38, the medical device 200 is inserted within the channel 32 of the agitator 30 until the medical device 200 actuates a tab or other suitable device (not shown) capable of generating an audible click or the like to indicate that the medical device 200 has been fully inserted within the channel 32 of the agitator 30. At this point, the medical device 200 is coupled to the coupling 38 inhibiting removal therefrom without additional manipulation of the medical device 200.

In step S310, the button 68 is depressed to cause the processor 62a to initiate the timing program stored on the memory 62b of the one or more drive circuits 62 and cause the LED 64 to illuminate to indicate that the cleaning process has begun, and cause the ultrasonic transducer 70 to generate ultrasonic energy that is propagated through the channel 32 of the agitator 30 to agitate or otherwise clean the medical device 200. In step S312, after a predetermined amount of time has elapsed, the timing program causes the LED 64 to shut off (e.g., stop illuminating light) or change color (e.g., from red to green, etc.) and cause the ultrasonic transducer 70 to terminate the generation of ultrasonic energy. At this point, in step S314, the medical device 200 is rotated to disengage from the coupling 38, and thereafter, is removed from the channel 32 of the agitator 30, and thereafter, from the aperture 32a, at which point the medical device 200 has been cleaned or otherwise disinfected and is once again ready to be utilized during a subsequent surgical procedure. After the cleaning process has been completed, in step S316, the pouch 20 and the agitator 30 are discarded.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments.

Throughout this description, the term “proximal” refers to the portion of the device or component thereof that is closer to the clinician and the term “distal” refers to the portion of the device or component thereof that is farther from the clinician. Additionally, in the drawings and in the description above, terms such as front, rear, upper, lower, top, bottom, and similar directional terms are used simply for convenience of description and are not intended to limit the disclosure. In the description hereinabove, well-known functions or constructions are not described in detail to avoid obscuring the disclosure in unnecessary detail.

What is claimed is:

1. A disposable cleaning system for a medical device, comprising:
a pouch having an interior surface defining a cavity; and
an agitator disposed within the cavity of the pouch, the agitator having an interior surface defining a channel along a length thereof, an exterior surface and the interior surface of the agitator defining an aperture in open communication with the channel, wherein the aperture is configured to slidably receive a portion of a medical device therethrough and permit the medical device to be advanced within the channel to disinfect the portion of the medical device disposed within the channel.

2. The disposable cleaning system according to claim 1, wherein the agitator includes a spiral configuration terminating at closed distal end portion.

3. The disposable cleaning system according to claim 2, wherein the agitator is formed from a resilient material, such that as a medical device is advanced within the channel, the agitator transitions from the spiral configuration to a configuration conforming to a profile of the medical device.

4. The disposable cleaning system according to claim 1, wherein the channel of the agitator includes a chemical disinfectant disposed therein.

5. The disposable cleaning system according to claim 1, wherein the aperture includes a penetrable seal configured to be penetrated by a portion of a medical device.

6. The disposable cleaning system according to claim 1, wherein the aperture includes a self-closing gland configured to be penetrated by a portion of a medical device.

7. The disposable cleaning system according to claim 1, further including a fluid reservoir disposed on the exterior portion of the agitator, the fluid reservoir having an inner surface defining a cavity having a chemical disinfectant disposed therein, wherein the cavity of the fluid reservoir is in fluid communication with the channel of the agitator.

8. The disposable cleaning system according to claim 7, wherein the cavity of the fluid reservoir includes a burst seal disposed therein configured to selectively permit the flow of the chemical disinfectant from the cavity of the fluid reservoir into the channel of the agitator.

9. The disposable cleaning system according to claim 1, wherein the interior surface of the channel includes a plurality of protuberances disposed thereon configured to contact an exterior surface of the medical device as the medical device is advanced within the channel.

10. The disposable cleaning system according to claim 1, wherein the aperture of the agitator includes a coupling configured to selectively retain a portion of the medical device and inhibit removal of the medical device from the agitator.

11. A disposable cleaning system for a medical device, comprising:
a pouch having an interior surface defining a cavity;
an agitator disposed within the cavity of the pouch, the agitator having an interior surface defining a channel along a length thereof, an exterior surface and the interior surface of the agitator defining an aperture in open communication with the channel, where the aperture is configured to slidably receive a portion of a medical device therethrough and permit the medical device to be advanced within the channel to disinfect the portion of the medical device disposed within the channel; and
a controller operably coupled to a portion of the agitator, the controller comprising:
a drive circuit;
an electrical switch in electrical communication with the drive circuit; and
a status light in electrical communication with the drive circuit.

12. The disposable cleaning system according to claim 11, further including a fluid reservoir disposed on the exterior portion of the agitator, the fluid reservoir having an inner surface defining a cavity having a chemical disinfectant disposed therein, wherein the cavity of the fluid reservoir is in fluid communication with the channel of the agitator.

13. The disposable cleaning system according to claim 12, wherein the cavity of the fluid reservoir includes a burst seal disposed therein configured to selectively permit the flow of the chemical disinfectant from the cavity of the fluid reservoir into the channel of the agitator.

14. The disposable cleaning system according to claim 11, wherein the controller further includes an ultrasonic transducer electrically coupled to the drive circuit, the ultrasonic transducer operably coupled to the channel of the agitator such that ultrasonic energy is permitted to propagate through the channel.

15. The disposable cleaning system according to claim 11, wherein the controller further includes an ultra-violet (UV) light source electrically coupled to the drive circuit, the UV light source operably coupled to the channel of the agitator such that UV light is permitted to propagate through the channel.

16. The disposable cleaning system according to claim 15, wherein the inner surface of the agitator includes a reflective coating disposed thereon.

17. A method for cleaning a medical device, comprising:
breaking a seal of a pouch having an inner surface defining a cavity;
advancing a distal end portion of a medical device through an aperture defined in an agitator, the agitator disposed within the cavity of the pouch;
further advancing the medical device through the aperture and into a channel defined by an inner surface of the agitator;
agitating the medical device within the channel to clean the medical device; and
retracting the medical device from the channel and aperture of the agitator.

18. The method according to claim 17, further including deforming a fluid reservoir having a chemical disinfectant disposed therein to cause the chemical disinfectant to transfer from the fluid reservoir and into the channel of the agitator to clean the medical device.

19. The method according to claim 17, wherein advancing the medical device within the channel of the agitator causes the agitator to transition from a spiral configuration to a configuration conforming to a profile of the medical device.

20. The method according to claim 17, wherein agitating the medical device within the channel causes an exterior surface of the medical device to abut a plurality of protuberances disposed on the interior surface of the channel to clean the medical device.
